(19) United States
(12) Patent Application Publication
(10) Pub. No.: US 2022/0347835 A1
(43) Pub. Date: Nov. 3, 2022

(54) REMOTE-CONTROLLED VEHICLE FOR OPERATIONS IN THE EXTREME CONDITIONS

(71) Applicant: DOK-ING d.o.o., Zagreb (HR)

(72) Inventor: Vjekoslav Majetic, Zagreb (HR)

(21) Appl. No.: 17/245,343

(22) Filed: Apr. 30, 2021

(51) Int. Cl. B25J 5/00; G05D 1/00; G05D 1/02; A62C 27/00; G01N 1/24; G01N 1/14; G01N 1/04; A61L 2/18

(52) U.S. Cl. CPC B25J 5/005; G05D 1/0011; G05D 1/0094; G05D 1/0231; A62C 27/00; G01N 1/24; G01N 1/14; G01N 1/04; A61L 2/18; G05D 2201/0207; F24H 7/002

(57) ABSTRACT

In this invention it is disclosed a remote-controlled vehicle for operations in the extreme conditions (10) which comprises a base vehicle (100), a gripper (200), a manipulator arm (300), a mission module wherein base vehicle comprises front (110) and rear part (120), wherein on the front part of the base vehicle gripper is mounted and on the rear part of the base vehicle the manipulator arm is mounted and when in operation, the gripper and the manipulator arm are remotely operated. Also, it is disclosed whole system for remote operations comprising said remote-controlled vehicle and a control centre comprising a set of graphical user interfaces (GUI) and video displays (VD) arranged so that two way communication between the vehicle and control centre is established and wherein from GUI person can control and operate with the vehicle.

### FIELD OF THE INVENTION

The present invention relates to remote-controlled vehicles suitable to operate in extreme conditions, especially environments with chemical and/or biological and/or radiological and/or nuclear threats and pollution.

### Technical Problem

There exist different types of natural and anthropogenic threats, especially coming from Chemical, Biological, Radiological and Nuclear (CBRN) materials or weapons and technological accidents in industrial and military complexes with serious CBRNe consequences causing great damage to people, environment and property. With this invention the inventor has a goal to develop a new means and technologies capable of eliminating CBRN threats without risk of human lives. The goal set up by inventor was to use a remote-controlled vehicle to fight against all these threats, thus completely eliminating human staff needed to operate vehicle in environment with direct life-threatening situations, but the vehicle is operated by humans at a safe distance from dangerous conditions.

Further goal was to design completely functional system with a prototype of the remote-controlled platform designed for detection (includes tasks of surveillance, reconnaissance and survey), identification, monitoring and elimination of hazards in conditions of chemical, biological, radiological, nuclear and explosive threats.

### PRIOR ART

As the prior art there are many patent applications for remote operated vehicles and remote-control systems for vehicles, robots and manipulator. Generally, in prior art i.e., in patent application US20180074490A1 Apparatus and method for vehicle remote controlling and remote driving system it is disclosed a vehicle remote control device and system, and a method for remote driving. Disclosed vehicle remote control system includes a remote driving vehicle transmitting driving information including images of a front, a rear, and sides, and path information of a vehicle, and vehicle condition information including a vehicle speed, an steering angle, front and rear pressure, a body tilt, an engine condition to a remote control platform, and receiving an ECU (Engine Control Unit) control signal from the remote control platform, thereby remotely running according to the control signal; and a vehicle remote control platform receiving the driving information from the remote driving vehicle, and remotely controlling the vehicle drive according to the received information. Claimed method does include other vehicle parameters too, but does not mention anything related to extreme conditions, neither firefighting nor decontamination. In patent CN207913067U Petrochemical industry Multifunctional firefighting robot and robot fire-fighting system it is disclosed a firefighting track-wheel type structure robot with fire water monitor for fire-fighting operation, including communication module for communication with control centre of robot, video mode identification mould for obtaining the photographing module of ambient image and carrying out pattern-recognition to acquired image block with All-in-One gas sensing module. This robot does not include decontamination function. Patent RU2580779C2 Mobile robotic fire extinguishing system discloses the mobile robotic fire extinguishing complex, which is a self-propelled vehicle and containing a body mounted on the chassis, on which is mounted a power plant, an on-board remote control and radio telemetry system, a fire extinguishing system, an on-board video surveillance system, as well as a special kit equipment consisting of a bulldozer blade and a water barrel monitor (fire monitor) with remote control. Further it discloses the power part including a diesel engine, hydraulic pumps, actuators and valves, fuel and oil tanks and a battery. The fire extinguishing system contains: fire equipment, communications for the supply of extinguishing agents, a centrifugal water pump with a hydraulic drive, connecting pipelines, water tanks and a foaming agent. Also disclosed is the remote-control system via the radio channel consists of an on-board control device with an antenna, is equipped with an autonomous wearable operator control panel (manipulator) and a mobile control centre. But, also, there is no decontamination disclosed. In U.S. Pat. No. 8,973,671B2 Smart compact indoor firefighting robot for extinguishing a fire at an early stage it is disclosed a firefighting robot comprising a robot platform having a double thermal insulation structure with a cooling system between a first and a second thermal structure of the double thermal insulation structure, the first and the second thermal structures each being comprised of a material with a low thermal conductivity and a strong thermal shock resistance with an extinguishing system platform and an extinguishing system, a driving camera mounted to a central front portion of the robot platform, a computing means programmed for the robot to analyse and fight fires and a remote control device adapted for controlling the computing means. This firefighting robot neither comprises decontamination system.

The search of PATENTSCOPE, USPTO Patent Database and ESPACENET did not reveal any patents or patent-pending applications comprising remote controlled vehicle comprising firefighting and decontamination subsystems with environmental sampling, nor a system with comprising the remote-controlled platform designed for detection (with surveillance, reconnaissance and survey), identification, monitoring and elimination of hazards in conditions of chemical, biological, radiological, nuclear and explosive threats.

### BRIEF SUMMARY OF THE INVENTION

The purpose of this remote-controlled vehicle for operations in the extreme conditions is operation in human hazardous environments and operation in extreme environment such as, conditions of open flame and high temperatures, explosive atmospheres (detection only), atmospheres with high concentrations of flammable, explosive, CBR contaminants and toxic industrial materials and as well as other CBRNe threats and terrorism threats in which responders cannot survive. To operate such a vehicle there is a complete system comprising base vehicle, CBRNe mission module comprising decontamination and firefighting, front tool with gripper, rear attachment with manipulator arm, remote control system, control centre solution, autonomous kit, environmental sensors, CR detectors and sampling system. The mission module is attached on top of the base vehicle. The front tool and manipulator arm are attached to the base vehicle. The sampling subsystem is attached to the manipulator arm and mission module. System capabilities include firefighting, decontamination, detection, identification and monitoring in hazardous environments, obstacle removal and sampling.

In this invention it is claimed a remote-controlled vehicle for operations in the extreme conditions characterised it that it comprises a base vehicle, a gripper, a manipulator arm, a mission module wherein base vehicle comprises front and rear part, wherein on the front part of the base vehicle gripper is mounted and on the rear part of the base vehicle the manipulator arm is mounted and when in operation, the gripper and the manipulator arm are remotely operated. Further the vehicle may comprise a sampling subsystem, environmental sensors and computed radiography (CR) detectors wherein the mission module is attached on top of the base vehicle, the sampling subsystem is attached partially to the manipulator arm and partially to the mission module, and environmental sensors and CR detectors are installed partially on mission module and partially on manipulator arm. The mission module might comprise a CBRN filter, water tanks, sprinklers and cooling air intake, wherein sprinklers are mounted above the cooling air intake and wherein when the vehicle in operation water from water tanks is sprinkled over intake air in cooling air intake. Further mission module might comprise electrical heaters built inside water tanks wherein the power for the heaters is provided from the base vehicle. A firefighting system installed on this vehicle comprises a high pressure water pump, a water cannon and a fire-fighting water tank and a foam tank positioned at the back of the mission module, wherein said fire-fighting water tank is equipped with quick coupling interface for water intake from the outside source, the water cannon being equipped with an auto-regulated pressure nozzle with ability to adjust the shape of the water jet. Also, a decontamination system comprises a decontamination emulsion mixer, at least one decontamination agent container and a tank positioned at the back of the mission module wherein the decontamination emulsion mixer a decontamination agent from decontamination agent container is remotely controlled. There are multiple decontamination nozzle network distributed on said mission module wherein, when claimed vehicle in operation, decontamination emulsion might be sprayed over the vehicle's exterior surfaces. The base vehicle might further comprise a hydraulic pump and the gripper comprises rotating jaws, lights and tool camera wherein the rotating jaws are powered by the hydraulic pump within the base vehicle and the gripper further comprises a blade. The manipulator arm might comprise a swivel stand and three rotational arms wherein swivel stand provides 360 degrees non-continuous rotation and wherein the manipulator arm is hydraulically operated, and in one further embodiment the manipulator arm comprises a gripper tool attached at the end of the manipulator arm, wherein the gripper tool is powered and comprises two additional rotating joints. Also, the manipulator arm might further comprise chemical warfare agent (CWA) detector, toxic industrial chemicals (TIC) detector, visual camera, shortwave and longwave thermal camera, temperature sensor and microphone.

Concerning firefighting, the system is able to perform firefighting operations with its firefighting system and blade-gripper front tool. Decontamination capability is provided by integration of high-pressure cleaning and system for applying decontamination liquid. System will provide support for hand-held decontamination.

Detection, identification and monitoring in hazardous environments is provided with video system, sensors and CR detectors. The system is able to detect, monitor and work in atmospheres with reduced oxygen and is able to detect explosive atmosphere, oxygen and temperature. Obstacle reduction/removal is possible with the usage of front tool. The vehicle is able to collect samples of solid, liquid or gaseous matters. Sampling capability is performed by gripper attached to the manipulator arm in the rear of the system. Sampling tubes are carried in the dedicated container located on the upper rear part of the mission module. Container is sealed, has remotely controlled hatch, secured against accidental opening. Container decontamination is done from outside. For sample analysis, tubes are manually extracted from the container, stacked in the portable carrier and carried to the laboratory.

Further, the vehicle is able to move in rough terrain and is able traverse 35 deg longitudinal incline and 25 deg side slope incline. The speed to move on horizontal road is 28 km/h, gross vehicle weight is planned to be up to 17.000 kg, include water, firefighting foam and decontaminating powder.

The system has a hybrid (Diesel-electric) powertrain. A set comprising a diesel engine, an electric generator and the most of the cooling system (Power Pack) is designed to be detachable from the machine and replaceable. The vehicle is able to provide energy from diesel fuel and electric energy from the batteries while various energy consumption scenarios are possible, some of them listed in Table 1. and Table 2. The vehicle is able to operate at full power with environmental temperatures of up to 50° C., and at ambient temperatures above 50° C. the vehicle works with reduced power. At temperatures above 65° C. vehicle uses only battery power and with reduced performance for limited amount of time that temperature rise in components allows, further cooling fans are disengaged and system with dew nozzles are activated.

TABLE 1
Working Scenarios with energy demand calculation, diesel fuel

| | | | AMOUNT OF ENERGY |
|---|---|---|---|
| MODE | SCENARIO | SCENARIO CHARACTERISTICS | AT SOURCE |
| USAGE | Drive on horizontal | 15 km/h (Drive Speed) | 10 L (Fuel) |
| OF | sandy ground | 110 kW (Power) | 28 kWh |
| DIESEL | | 15 min (Time) | |
| FUEL | Drive on sandy | 7.5 km/h (Drive speed) | 11 L (Fuel) |
| | ground with 15 deg | 135 kW (Power) | 34 kWh |
| | incline | 15 min (Time) | |
| | Drive on horizontal | 20 km/h (Drive speed) | 5 L (Fuel) |
| | asphalt | 58 kW (Power) | 15 kWh |
| | | 15 min (Time) | |
| | Drive on asphalt | 10.5 km/h (Drive speed) | 11 L (Fuel) |
| | with 15 deg incline | 135 kW (Power) | 34 kWh |
| | | 15 min (Time) | |
| | Working cycle | 16.8 t (Vehicle mass Caterp.) | 20 L (Fuel) |
| | according to | 166 hp (Engine Power) | 60 kWh |
| | Caterpillar D6N | 60 min (Time) | |
| | dozer | | |
| | Battery pack | 80 kW (Charging power) | 27 L (Fuel) |
| | charging | 60 min (Time) | 80 kWh |
| | | 1x Charging cycle | |
| | Fire extinguishing | 90 kW (System power) | 90 L (Fuel) |
| | | 3 h (Time) | 270 kWh |
| | Work with tools | 13 kW (Required power) | 10.8 L (Fuel) |
| | (according to | 5 h (Working time) | 32.5 kWh |
| | Palfinger telescopic | 50% (duty cycle) | |
| | boom) | | |
| | Total energy | This data does not include the | The Amount of energy |
| | consumption in | bellow scenario, supply of | 180 L (Fuel) |
| | Diesel scenario | external consumers. The | 540 kWh |
| | | Assumption is that it is possible | Energy amount before |
| | | to replenish the fuel when the | the diesel engine |
| | | machine is working as a power | 225 L Fuel (η_{D} = 0.8) |
| | | source | 675 kWh (η_{D} = 0.8) |

TABLE 2
Working Scenarios with energy demand calculation, batteries

| Energy | | | AMOUNT OF ENERGY |
|---|---|---|---|
| Used | SCENARIO | SCENARIO CHARACTERISTICS | AT SOURCE |
| DEPLETION | Drive on horizontal | 15 km/h (Drive speed) | 28 kWh |
| OD | asphalt | 42 kW (Required Power) | 9.2 L (Fuel equivalent) |
| BATTERIES | | 40 min (Time) | |
| | Working cycle | 16.8 T (Vehicle mass) | 15 kWh |
| | according to | 166 hp (Engine power) | 5 L (Fuel equivalent) |
| | Caterpillar D6N | 15 min (Time) | |
| | dozer | | |
| | 20 L/h (Fuel per | | |
| | hour) | | |
| | Fire extinguishing | 90 kW (System power) | 22.5 kWh |
| | | 15 min (Time) | 7.5 L (Fuel equivalent) |
| | Work with tools | 13 kW (Required power) | 13 kWh |
| | (according to | 2 h (Tool working time) | 4.2 L (Fuel equivalent) |
| | Palfinger telescopic | 50% (duty cycle) | |
| | boom) | | |
| | “Standby” | 3 kW (Equipment power | 3 kWh |
| | “Silent Watch” | consumption) | 1 L (Fuel equivalent) |
| | (Vehicle and tools at | 60 min (Time) | |
| | rest | | |
| | Total energy | | OUTPUT ENERGY |
| | consumption in | | 82 kWh (90%) |
| | Battery scenario | | 27 L (Fuel) |
| | | | Amount of energy at |
| | | | source (Batteries) |
| | | | 82 kWh (η_{E} = 0.85) |
| | | | 28 L Diesel fuel equivalent |
| | | | (η_{E} = 0.85) |

Remote control provides data link to and from the vehicle and video link, from the vehicle, in distance of 1500 m LOS (line of sight). Remote control system is divided in three levels:
Level 1.
   control of the machine and all of the subsystems of the machine with hand held controller without integrated video
   controller displays all feedback data from the machine needed for the operational use of the machine
Level 2.
   control of the machine through the video case with the integrated OCU (operator control unit)
   integrated OCU is implemented with the joysticks enabling operator to operate machine in full CBRN protective equipment (including rubber gloves)
Level 3.
   control of the machine with the use of the command and control centre (CCC)

In hazardous environment the vehicle functions as an autonomous system. The vehicle is capable of doing patrolling, way pointing, returning home and obstacle avoidance. Obstacle avoidance is based on input from LIDAR.

The base vehicle is tracked platform used for providing mobility and electrical power to various elements and modules this vehicle consists of and the base vehicle is usually powered by an electric generator connected to a diesel engine. The system also has rechargeable energy storage system (RESS). The powertrain of vehicle is completely electric and consists of two electric motors, one for each track.

The base vehicle interior is dived in three sections, front, middle and rear section. Front section contains hydraulic subsystem, middle section contains battery pack and electronics and rear section contains powerpack. Front and middle sections are pressurized. Rear section, containing diesel engine and cooling system is open to the atmosphere. In order to evacuate water that might otherwise flood the rear compartment during rain, washing or sprinkler operation, the rear compartment is equipped with bilge pump that can be turned on manually by the user or via the remote control (OCU) or from VCP.

Hydraulic subsystem provides power for cooling fans, front tool and manipulator arm. The output of electric drive motor is connected to dual-gear automatic transmission. The transmission has a high-speed gear and high-torque gear. The high-torque gear is used in conditions where higher torque is needed, such as for example pulling/pushing loads, climbing uphill, going through muddy terrain, etc.

The base vehicle is equipped with tracks. Sprockets, located at the upper rear part of the tracks are attached to the transmission output. Tensioning wheels are located at the front upper part of the tracks. Track tensioning is provided by grease filled cylinders. Upper part of each track is supported and guided by two support rollers. Damping of the pitch oscillations of the vehicle is provided by shock absorbers. First two and rearmost (fifth) pair of road wheels are equipped with shock absorbers. Further, the base vehicle is also equipped with slope sensor that measures vehicle roll and pitch angles. The purpose of this sensor is for both displaying the slope information to user as well as providing transmission controller with information for the purpose of estimating the braking forces on a downhill/uphill slope. This information is used to ensure that the maximum downslope speed is not exceeded, which equals the maximum power that can be attained with braking with vehicle maximum mass on slope.

Cooling system used in base vehicle is complex, multi-liquid system that is supplying coolants to every aspect of machine from power electronics, electric motors, hydraulics to diesel engine. The diesel engine, electric generator and most of the cooling system are integrated into a single unit designated the Power Pack. The Power Pack is designed to be detachable from the machine and replaceable in workshop. The vehicle is equipped with onboard charger.

The vehicle control panel (VCP) is an interface between user and the machine that conveys important parameters and machine status to user and also allows for some machine diagnostics and maintenance. The screens on VCP are divided into four groups: settings—used for setting up machine user settings, telemetry, diagnostics and maintenance.

From time to time, regarding on the vehicle usage, soot gets accumulated on the exhaust system walls which deteriorates the effectiveness of the aftertreatment system. The process of removing the soot from the exhaust wall is employed, known as aftertreatment regeneration.

The base vehicle also features warning system in form of a horn located at the back of the vehicle. In addition to diesel fuel, diesel engine also requires urea solution for aftertreatment also known as AdBlue.

Vehicle needs both hydraulic oil and coolant oil to function properly. Vehicle also requires coolant fluid (antifreeze) for operation. Coolant fluid tank is integrated into coolant system radiators in the back of the vehicle, on the Powerpack.

Gripper with or without a blade is the tool that is attached to the front of the base vehicle. It will provide the system with ability to manipulate and remove objects and obstacles from the operating area and lift and manipulate heavy loads. It will provide the user with the ability to use rotating jaws. Gripper is equipped with lights and tool camera and is powered by a hydraulic pump within the base vehicle.

The manipulator arm is attached to the base vehicle at the rear side, comprising a swivel stand and three rotational arms. Chemical warfare agents (CWA) and toxic industrial chemicals (TIC) detector, visual camera, shortwave and longwave thermal camera, temperature sensor and microphone are located on the manipulator arm and on the one rotational arm there are integrated high pressure cleaning system and system for applying decontamination liquid, wherein water is supplied from the mission module.

The manipulator arm provides following tasks:
point sensing, data acquisition and distribution with integrated cameras, sensors and detectors
CBR decontamination of facilities, combat and non-combat techniques and partial self-decontamination
CBRe sampling and sample storage
performing actions such as opening a door (window), penetrate through lighter partitions such as glass, sheet metal and wall made of gypsum plasterboard and siporex, etc
extinguishing small fires independently of the fire system
additional irrigation with CBRN decontamination system when entering the high temperature and open fire zone
automated gripping of sampling tubes from a transport crate and their automatic return to a transport crate after sampling is completed
is used for partial decontamination (high pressure cleaning and decontaminant spreading) of upper and side surfaces of the system
is able to operate in coordinate mode with automated joint positioning

The manipulator arm is able to fold when not in use in order not to protrude from the system silhouette. When working in automated mode manipulator arm has predefined movement between predefined positions. This is important to avoid system external surfaces and parts. When not in automated mode the control of the manipulator arm is manual. In manual mode manipulator arm is supported visually via video cameras.

A CBRN mission module (further mission module) is usually equipped with 4 eyelets, 2 at the front and 2 at the back of the module, for lifting the module with crane. Before lifting, all tanks inside the module have to be emptied. The mission module is attachable/detachable to/from the base vehicle. A crane has to be used to position the mission module on top of the base vehicle. After positioning, the mission module must be secured with bolts on all 4 connecting points. In order to transfer the energy from the base vehicle 2 power cables must be connected at the front right end of the vehicle. The mission module is detached from the base vehicle in reverse order. Further the mission module is equipped with sprinklers mounted above the cooling air intake wherein sprinklers are used for intake air cooling and decontamination There is an option for mission module for user to activate prevention of entrance of contaminated atmosphere via operator control unit (OCU) so the contaminated atmosphere cannot enter into the system.

The mission module superstructure is resistant to explosion blast, infantry fire and fragmentation according to STANAG 2920 and MIL-STD-662F: 1.1 g FSP: V50 of 500-600 m/s and fragmentation resistance V50>1000 m/s. These terms also apply to some equipment mounted outside of the mission module which include: parts of the fire fighting and decontamination system that are permanently attached to the superstructure, firefighting and decontamination piping system and firefighting and decontamination system nozzles. Some of the equipment have partial resistance to explosion blast, infantry fire and fragmentation according to aforementioned standard which include: video cameras—positioned inside housing with an opening for camera lens, environmental sensors and CR detectors—positioned inside housing with an opening for environmental readings and mission module lights and signalization—positioned inside housing with an opening for light lenses.

The mission module might have electrical heaters built inside water tanks to prevent freezing of the water while the vehicle is exposed to water freezing temperatures. Further the mission module might be equipped with a firefighting system comprising a water tank, a foam tank, an appropriate quick coupling interface for water intake from the outside source (hydrant, tank trailer, lake or other water sources), a water cannon and a high-pressure water pump. The firefighting system is remotely controlled via OCU with an option of fire fighting with water or water/foam mixture with a different foam dosage. The mission module might be equipped with appropriate interfaces (fast couplings) for transferring high pressure water to the manipulator arm and hand-held high-pressure water gun. The high-pressure water system for manipulator arm is remotely controlled via OCU, while the hand-held high-pressure water gun is enabled via OCU and then activated via VCP. Both systems can work simultaneously or separately.

Operator is able to turn on self-protection from the OCU. Water for self-protection is supplied from the main water tank and is functional as long as it is not turned off, or all the water has been used. OCU alarms the operator if any of the external ambient temperature sensors reads previously set temperature level. Alarm might be in the form of warning message and warning sound. IRI_HMI software (part of Command-and-control Centre solution) has functionality to warn the operator by sound, show the measured value and position of the sensor on the graphical interface and log the data. Self-protection system includes an option of injecting water spray in the air intake of the base vehicle cooling system for additional heat dissipation. This functionality can be (de)activated simultaneously with self-protection from high temperature or (de)activated separately from it.

Further mission module is equipped with a decontamination system comprising a tank positioned at the back of the mission module, with remotely controlled decontamination emulsion mixer, at least 2 additional doses of decontamination agent mounted on the mission module with a remotely controlled system for adding it to the decontamination tank. Further, the decontamination system might involve multiple separate systems that can work independently. Those systems include: partial self-decontamination, decontamination with manipulator arm at the back of the vehicle, decontamination of the horizontal surfaces at the front of the vehicle, decontamination with shower at the back of the vehicle, decontamination with portable shower and hand-held decontamination gun. The showers and the hand-held decontamination gun are enabled via OCU and then can be activated via VCP, while all the rest of the system is remotely controlled via OCU only. The decontamination system might have built-in heater that can heat up the decontamination emulsion. The fuel level of the heater tank is remotely monitored and the heater itself remotely controlled via OCU. The heater is remotely controlled and can be activated during vehicle operation. Partial self-decontamination system comprises multiple decontamination nozzle network distributed on the mission module that can decontaminate the surface of the vehicle that is inaccessible for partial self-decontamination with manipulator arm. In combination with manipulator arm, decontamination nozzle network can decontaminate most of the vehicle's surface except the water cannon stand and the front tool which is decontaminated with hand held decontamination gun plugged to the mission module interface. The mission module is equipped with appropriate interfaces (fast couplings) for transferring decontamination emulsion to the manipulator arm and hand-held decontamination gun. Partial self-decontamination with manipulator arm is an automated process that only requires remotely controlled activation/deactivation from the user. Two automatic mods are present: partial self-decontamination of the vehicle tracks and partial self-decontamination of the vehicle outer shell.

The partial self-decontamination process implies washing with high pressure water and dispersing the surface with the decontamination emulsion. Decontamination of the horizontal surfaces is a part of the decontamination system that is mounted on the front tool of the vehicle. The mission module is equipped with appropriate interfaces (fast couplings) for transferring decontamination emulsion to the front tool. Beside the partial self-decontamination, the manipulator arm is able to decontaminate buildings, fortifications and military/civilian machinery. Decontamination system includes decontamination of the gear and clothing of the first response team with mounted shower at the back of the vehicle. Also, portable shower can be mounted at the safe distance from the vehicle and plugged to the appropriate interface (same coupling as the hand-held decontamination gun) for decontamination of the personnel. Both systems can be activated individually or at the same time.

The powertrain space has an air filtration system located on the upper surface as a protection against contamination.

Control of this vehicle and all of the subsystems is with hand held controller without integrated video. Hand held controller will display all feedback data from the vehicle needed for the operational use of it. Control of this vehicle is via the command-and-control centre (CCC) by remote control.

Remote control system provides possibility to control the base vehicle, gripper, movement of the firefighting monitor, manipulator arm, firefighting system functions, decontamination system functions, camera system functions, mission module, power pack, autonomy and detection.

Remote Control system has an ability to display data about base vehicle, gripper, manipulator arm, firefighting system functions, decontamination system functions, camera system functions, mission module, power pack, autonomy and detection. Environmental sensorics and detection is divided in to point and ambient detection. Claimed vehicle is able to collect samples of solid, liquid or gaseous matters. This is done by gripper tool. The sampling tubes are carried in the dedicated container located on the rear upper part of the mission module. The container is hermetically sealed and decontamination resistant.

Sampling procedure is automated in a way that a container lid opens and arm is positioned to take particular sampling tube. The manipulator arm is manually controlled to the sampling place and during the sampling procedure. After sample is collected, contained in the tube, arm will return the tube into the bin and bin lid is closed.

The sampling system integrates:
gas, aerosol and dust sampling tubes
sampling tubes for liquid samples
solid sample sampling tubes

The sampling system works as follows:
gas, aerosol and dust sampling tube consist of a housing and an electric motor with a blower to generate pressure; electronic tube operation control; sets of sample adsorption filters (cellulose filter, silicate filter and activated carbon filter) and outlet valves on the side of the housing and the bottom of the tube with the inlet valve. The lower part is detachable from the upper case. It operates on the principle of blower creating flow through the valve and the adsorption of the sample on the filters.
the sampling tube for liquid samples consists of a housing and an electric motor with electronic tube operation control; reducer of electric motors; injector for the injection of fluid and the lower part of the tube with the inlet valve which is the sample carrier. The lower part is detachable from the upper case. It operates on the principle of pressure generated by the injector, and upon completion of sampling, the inlet valve immobilizes the sample between the top piston of the injector and the inlet.
solid sample sampling tube consists of a housing and an electric motor with electronic tube operation control; reducer of electric motors; roller shafts and drill bits with pockets inside the roller to hold and hold the bulk and solid specimen; the lower part of the tube with the lid at the end for sealing the sample tube. The lower part is detachable from the upper case. It operates on the principle of a rotary drill that drills and takes a sample, and the lower part of the tube moves upwards by pressing o the sample and opens the drill-drill, and after completion, by spring action returns to the basic position and closes the tube.

The container for housing the sampling tubes is intended to safely store the sampling tubes. It is located on the upper part of the superstructure, fastened to the superstructure, and the tubes in the crate are secured with supports. After sampling is completed, and after external decontamination (partial self-decontamination) of the container, tubes are removed from the container and transported to the laboratory. The capacity of the crate is 3×3 tubes, of which three tubes can be used to take a reference sample outside the contaminated area.

Upon sampling task, the operator puts the robotic arm into mode for autonomously taking the tube from the crate after selecting the type of sample. After the tube is taken autonomously, the operator manipulates the robotic arm and brings the tube to the sampling point, and upon sampling, returns the robot arm to the mode for autonomously storing the tube in the crate.

The control centre being part of the system is implemented as the set of graphical user interfaces, video displays, machine operator work place and commander work place wherein appropriate hardware is used for the communication and recording function. The meteorological station also might be the part of control centre. Some typical functions and displays present in the control centre are:
1) Display with telemetry from the machine
2) Display with video feedback from the machine
3) Display with user interface for H3D Radiological Imager
4) Display for geographic information system
5) Display for images from the LIDAR
6) Display for commander PC

### DESCRIPTION OF THE DRAWINGS

The accompanying drawings, which are referred to in the description and which form a part of the description, illustrate the best invention embodiment and help explain the basic principles of the invention.

FIG. 1. shows the remote-controlled vehicle for operations in the extreme conditions

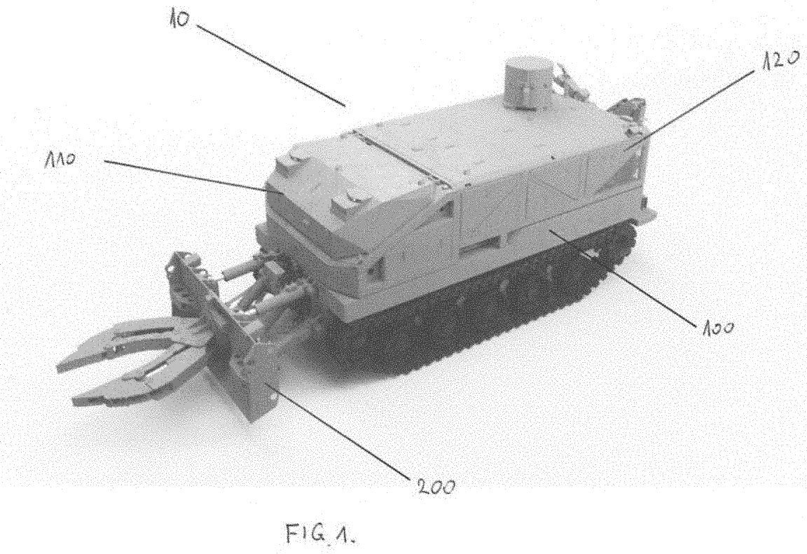

FIG. 2. shows enlarged view of the gripper

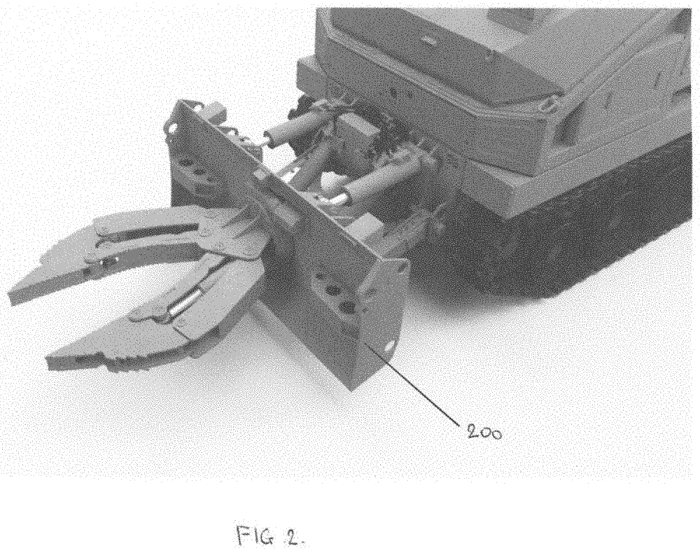

FIG. 3. shows rear view of the remote-controlled vehicle for operations in the extreme conditions

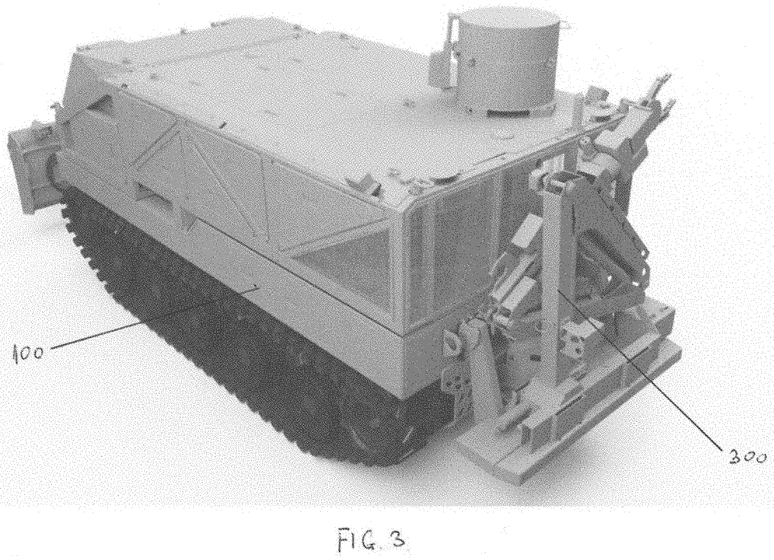

FIG. 4. shows Power Pack and base vehicle

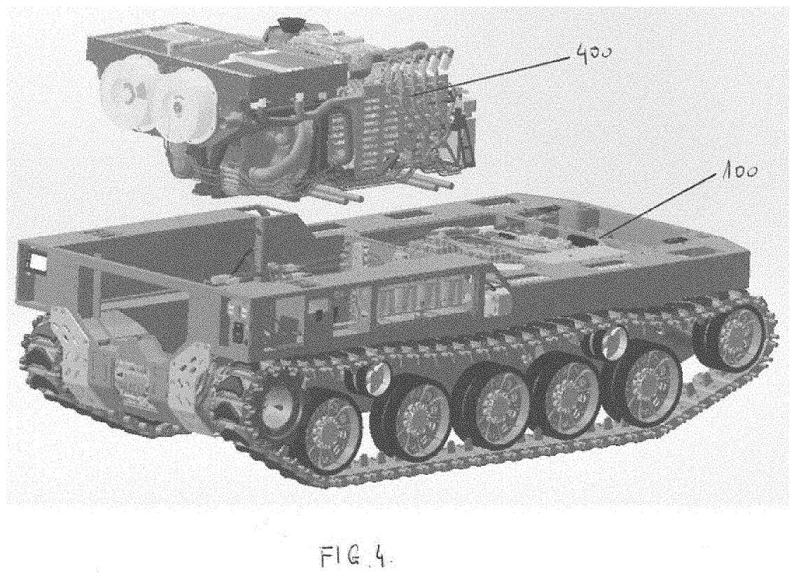

FIG. 5. shows system overview

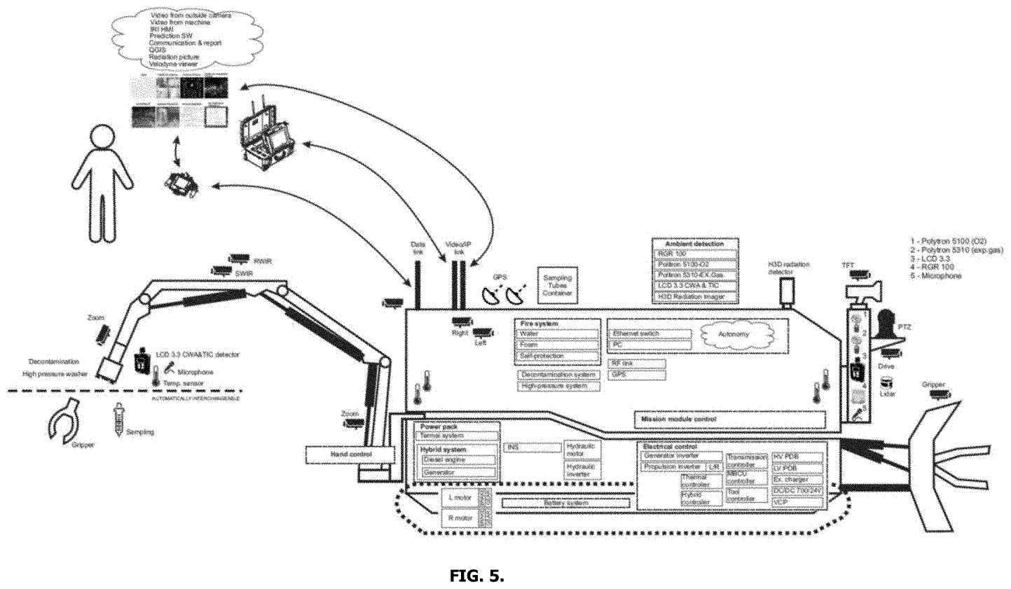

FIG. 6. shows the base vehicle control units

FIG. 7. shows macro operator control unit-top view

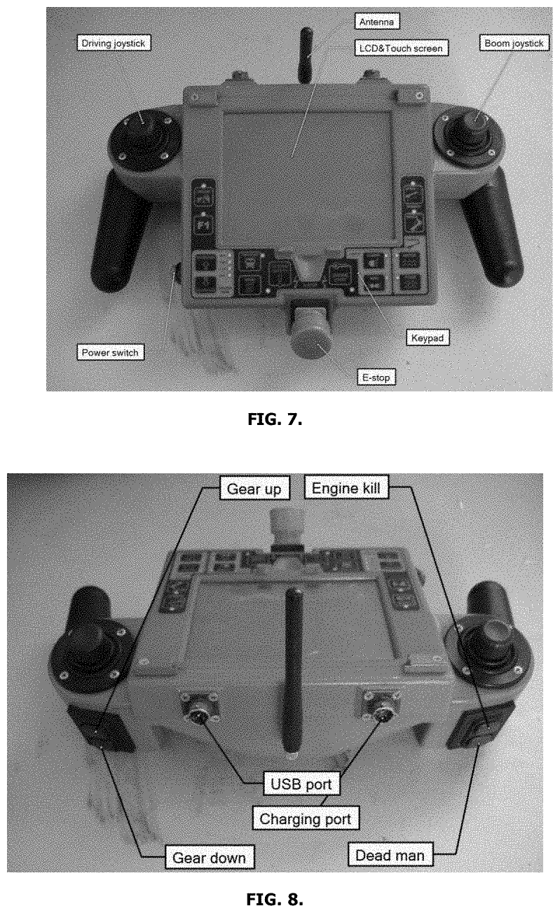

FIG. 8. shows macro operator control unit-front view

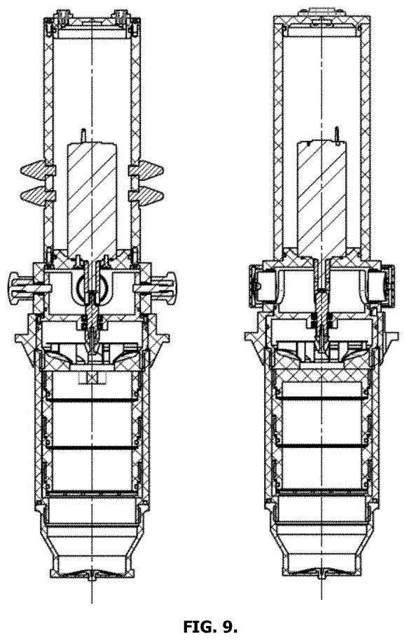

FIG. 9. shows gas sampling tube

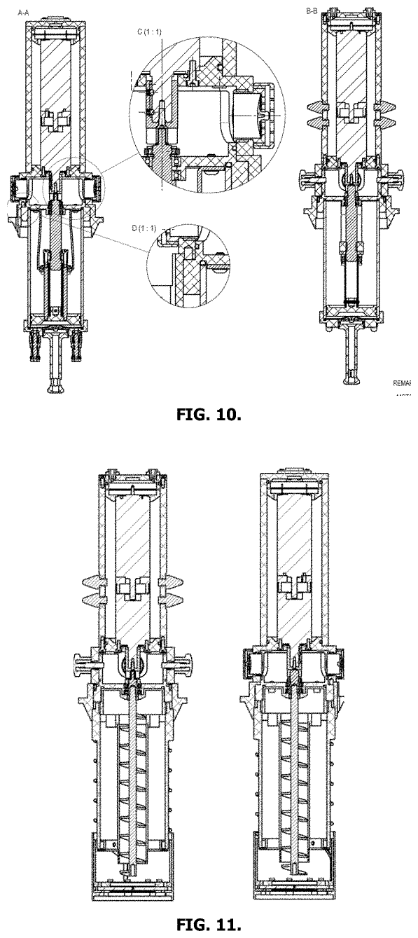

FIG. 10. shows liquid sampling tube

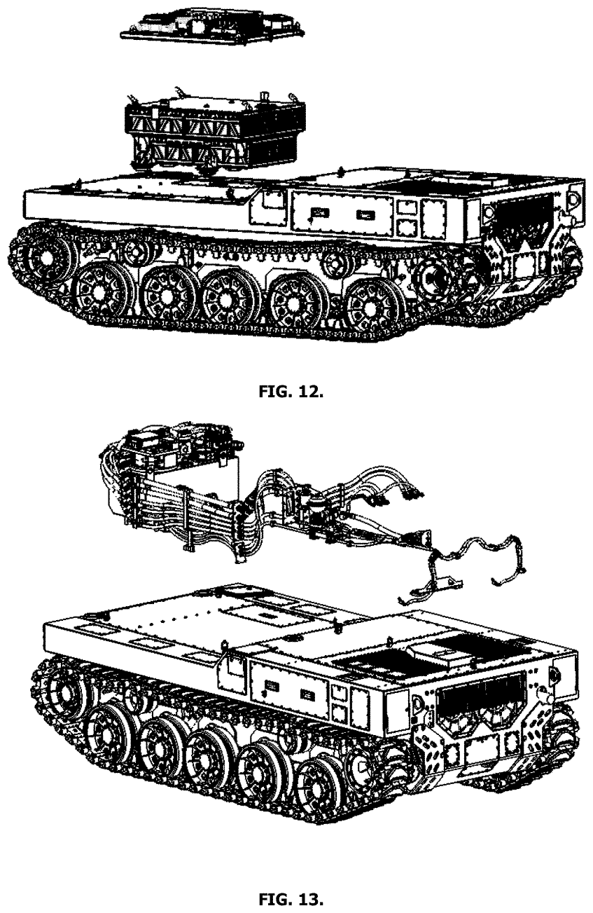

FIG. 11. shows solid sampling tube

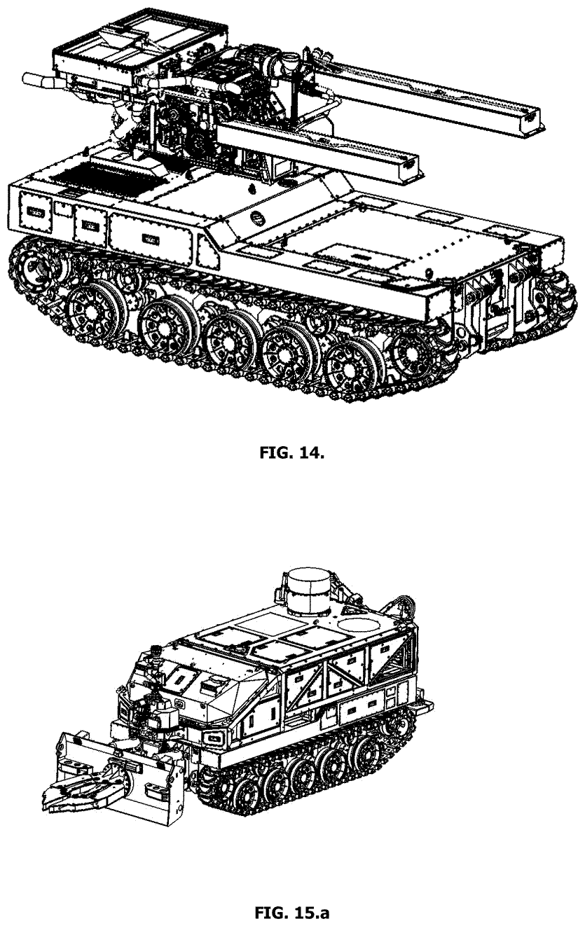

FIG. 12. shows another view of Power Pack and base vehicle

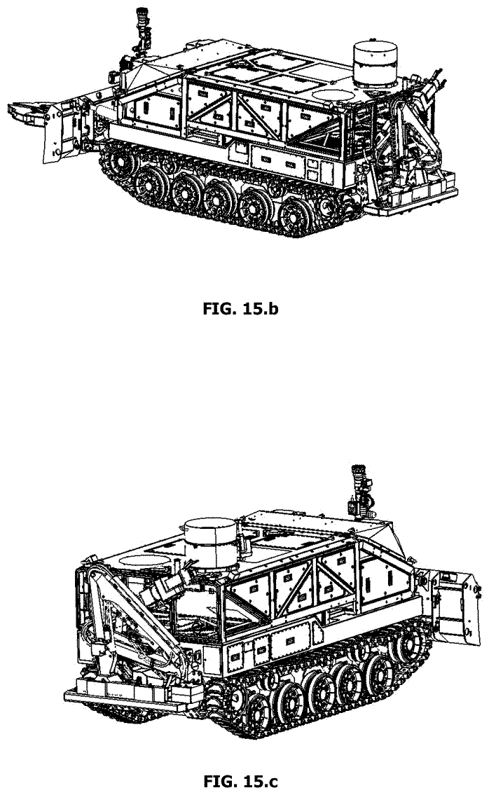

FIG. 13. shows base vehicle with installation module

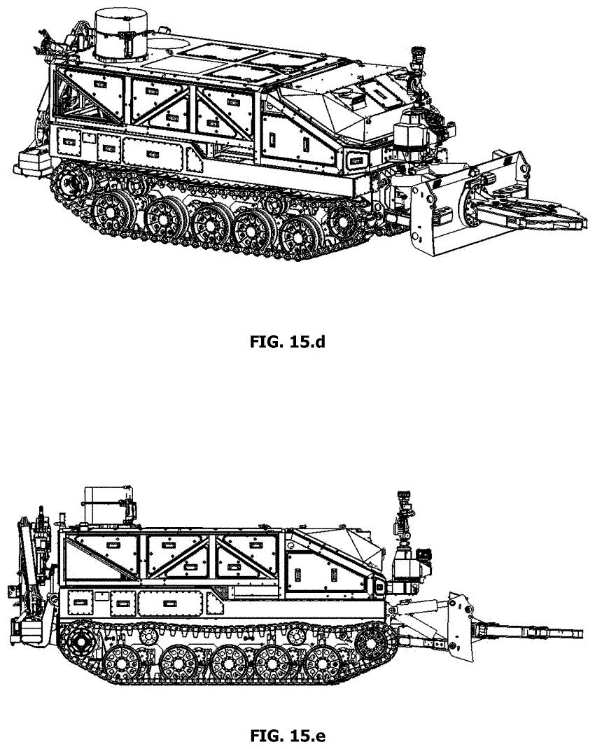

FIG. 14. shows another embodiment of the remote-controlled vehicle for operations in the extreme conditions

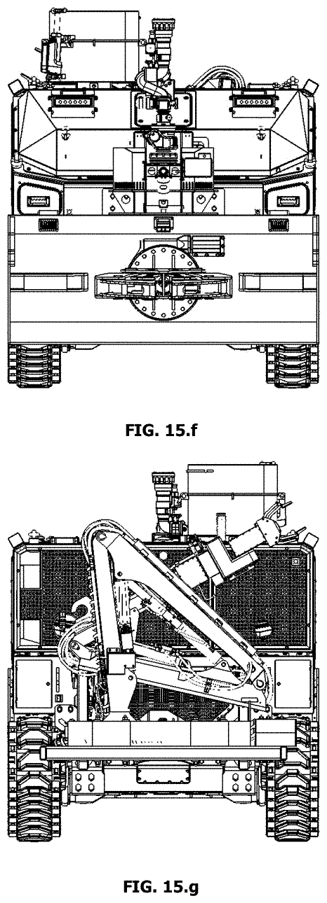

FIG. 15.a,b,c,d,e,f,g,h. shows different views of one embodiment of the remote-controlled vehicle for operations in the extreme conditions

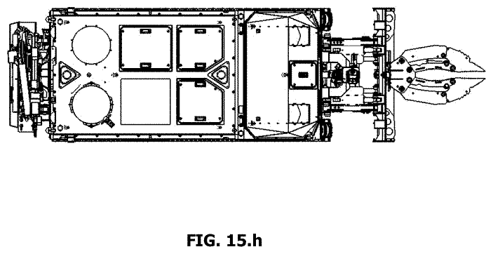

### A LIST OF REFERENCE SIGNS USED IN THE DRAWINGS

10— remote-controlled vehicle for operations in the extreme conditions
100—base vehicle
110—base vehicle front part
120—base vehicle rear part
200—gripper
300—manipulator arm
400—Power Pack

### THE LIST OF ABBREVIATIONS USED

CBRN—Chemical, Biological, Radiological and Nuclear

ECU—Engine Control Unit

CR—computed radiography

CWA—chemical warfare agent

TIC—toxic industrial chemicals

LOS—line of sight

OCU—operator control unit

CCC—command and control centre

RESS—rechargeable energy storage system

MBCU—Mobile Base Control Unit

HCU—Hybrid system Control Unit

TRCU—Transmission Control Unit

THCU—Thermal Control Unit

TCU—Tool Control Unit

Detailed Description of at Least One Way of Carrying Out the Invention

The base vehicle 100 is a welded metal structure made of steel sheets and profiles. For power is used an electric generator connected to a diesel engine with expected generator power of approx. 180 kW. Also, a rechargeable energy storage system (RESS) is employed comprising LiFePO4 battery packs with total energy capacity of around 92 kWh. There are two electric motors, one for each track, with combined peak power of up to 360 kW. The base vehicle 100 comprises base vehicle front part 110 and base vehicle rear part 120. Base vehicle interior is constructed in a way it is dived in three sections: front, middle and rear section. In the front section is hydraulic subsystem, in middle section is battery pack and electronics and in the rear section is powerpack. Front and middle sections are pressurized and isolated from the outside atmosphere.

A bilge pump is placed in the rear section and it can be turned on manually by the user or via the remote control (OCU) or from VCP with the function to evacuate water that might otherwise flood the rear compartment during rain, washing or sprinkler operation.

Hydraulic subsystem provides power for cooling fans, front tool and manipulator arm, and is arranged so that in typical embodiment it delivers 70 kW at 275 bars. The output of electric drive motor is connected to dual-gear automatic transmission. The transmission is with a high-speed gear and high-torque gear. The high-speed gear allows the maximum speed the vehicle can achieve at 28 km/h. Maximum force produced by vehicle in high-torque mode typically is over 150 kN, but its speed is limited to 14 km/h.

The base vehicle 100 is equipped with tracks and typically each track consists of 64 steel track shoes with rubber pads. Base vehicle undercarriage is equipped with 5 independently suspended road wheels on each side. Suspension is of torsion bar type with transversal torsion bars for each road wheel. Sprockets are located at the upper rear part of the tracks are attached to the transmission output. Tensioning wheels are located at the front upper part of the tracks. Track tensioning is provided by grease filled cylinders. Upper part of each track is supported and guided by two support rollers. Damping of the pitch oscillations of the vehicle is provided by shock absorbers. First two and rearmost (fifth) pair of road wheels are equipped with shock absorbers. Further, the base vehicle 100 is equipped with slope sensor that measures vehicle roll and pitch angles. Base vehicle's cooling system is designed to operate at ambient temperatures up to 50° C. without power reducing or up to 65° C. with reduced power. Cooling system is complex, multi-liquid system that is supplying coolants to every aspect of machine from power electronics, electric motors, hydraulics to diesel engine. The cooling system has two high performance, hydraulically powered axial fans for forcing substantial air flow through coolant radiators. The cooling system is split into two sections, each with its own set of radiators and its own fan. In order to minimize the power consumption of the cooling system, which can go up to 80 kW, the sections are designed in such a way that one section is needed only when the diesel engine is running, while the other is necessary to run at all times.

The diesel engine, electric generator and most of the cooling system are integrated into a single unit (Power Pack) 400 arranged so it is detachable from the machine and replaceable in workshop.

The base vehicle has gripper tool in front and manipulator arm 300 back attachment to which it supplies 24V, CAN bus for control and hydraulic power. Base vehicle has top side interface for MM with high voltage DC electrical power, 24V DC auxiliary power and CAN buses for control.

Back of the machine has two user panels. Left panel is fitted with Vehicle Control Panel (VCP), start/stop button, emergency stop button, diagnostic connector, connector for wired control. The right panel is fitted with High-voltage main fuses, charger connector and NATO AUX power supply connector.

High-voltage main fuses are of MSD type (Manual Service Disconnect) meaning they have dual purpose. Primary purpose is protection of the battery pack from high currents in excess of 630 A. Secondary purpose is to allow manually disconnecting the battery pack high voltage from rest of the system in case of maintenance.

The vehicle is equipped with onboard charger with maximum charging power of 22 kW. Charging is done via standard IEC62196 EV charging socket. The socket is CCS (Combined Charging Standard) socket meaning it features both the AC and fast DC charging pins. Vehicle is designed to be able to charge from standard AC grid systems (220V/50 Hz single phase and three phase, 110V/60 Hz single and three phase). In case fast charging is necessary, vehicle is able to enter special mode where it utilizes diesel generator to charge the battery in 60 min. This mode is activated by the user via VCP or OCU.

AUX NATO power supply connector is primarily used for supplying high power 24V to external systems. Additionally, the connector can be used for supplying power to the 24V rail from external power source in case when, for example the AUX batteries have been drained.

The screens on VCP are divided into four groups, each of them displaying following data:
Settings
   Settings screen
      Radio link parameters (callbook, transmit power, channel, etc.)
      Parameter logging selection (select which parameters are to be logged)
      Track offset compensation L/R
Telemetry
   Main screen
      Battery status percent
      Diesel fuel level
      Diesel engine RPM
      Coolant system temperatures (×5)
      Radio link status
      AdBlue status
      Hydraulic oil level
      Hydraulic oil temperature
      Parking brake status (oil pressure)
   Status screen
      Machine status
      MBCU status
      HCU status
      TRCU status
      THCU status
      TCU status
      BMS status
      Mission module status
   Battery system screen
      Battery status (percent/kWh)
      Battery pack voltage
      Battery cell voltage (×198)
      Battery temperature MAX
      Battery temperature AVG
      Battery current
      Battery power
      BMS status
   Hybrid powertrain screen
      Diesel engine RPM
      Diesel engine coolant temperature
      Diesel engine inlet air temperature
      Diesel engine fuel level
      Diesel engine status
      Diesel engine oil level
      Diesel engine inlet air pressure
      Diesel engine alternator current
      Diesel engine battery voltage
      Diesel fuel temperature
      Aftertreatment temperatures
      Aftertreatment AdBlue level
      Aftertreatment urea quality
      Aftertreatment system status
      Generator temperature
      Generator inverter temperature
      Generator inverter bus voltage
      Generator torque
      Generator inverter status
      Generator inverter faults
      HCU status
   Cooling subsystem (thermal management) screen
      Fan 1 RPM
      Fan 2 RPM
      HV pump 1 RPM
      HV pump 2 RPM
      LV pump 1 RPM
      LV pump 2 RPM
      Coolant upstream temperature (5×)
      Coolant downstream temperature (5×)
      Water/glycol coolant level (×2)
      Oil coolant level (×2)
      THCU status
   Transmission subsystem screen
      Current gear
      Hydraulic accumulator pressure
      Hydraulic oil temperature
      Motor RPM (L/R)
      Track RPM (L/R)
      Brake pressure (L/R)
      Parking brake status (L/R)
      TRCU status
   Tool system screen
      Hydraulic pump RPM
      Hydraulic oil temperature
      Hydraulic oil pump pressure
      Tool valves status (×6)
      Hydraulic oil level
      Front tool status
      Rear tool status
      TCU status
   Miscellaneous screen
      Ambient air pressure (from engine)
      Ambient air pressure (from HPT sensor)
      Ambient air temperature
      Ambient air humidity
      Internal air pressure
      Internal air temperature
      Internal air humidity
      Slope sensor tilt
      Slope sensor pitch
   Log screen
      Logged parameters
      Diagnostic errors
      Diagnostic faults
   Mission module (CBRNE) screen
Diagnostics
   Hybrid control system test
      Procedure:
         Start diesel engine
         Regenerate 80% of rated power using generator
         Run test for 60 seconds maintaining pre-set electrical power level
         Turn off engine
         If the achieved power level is within +/−5% the whole time, the test is passed
      Purpose:
         Since user has no direct control of diesel engine this is a way of testing that the hybrid powertrain works
   Cooling subsystem
      Procedure:
         Turn ON hydraulic pump
         Turn ON hydraulic fan for diesel engine cooling
         Turn ON hydraulic fan for rest of system
         Turn ON HV electrical pumps
         Turn ON LV electrical pumps
         Maintain operation for 120 seconds
         Turn OFF pumps
         Turn OFF fans
         Turn OFF hydraulic pump
         If the measured fan RPMs were nominal and temperatures of coolants are equal to ambient air+/−5 C the test is passed
      Purpose:
         Since the user has no direct control over cooling fans and they are not engaged automatically this is a way of testing that the cooling system works
Maintenance
   Aftertreatment system regeneration
      Turn ON diesel engine
      Ramp up RPM and power to heat up the exhaust system
      Run for 30 minutes to incinerate the residue on the exhaust walls
      Turn OFF diesel engine
      Alert user that the procedure has been completed

To remove the soot from the exhaust wall regeneration process is employed with predefined levels of the exhaust system clogging in the range from 0 to 5 (ZONE 0-5), where 0 means no clogging while 5 is clogging level where diesel operation is not possible without regeneration. Regeneration should start at the time of earliest convenience starting from ZONE 2. Information regarding the aftertreatment status is available via indicators present on both the VCP and OCU. This aftertreatment regeneration procedure can also be initialized from both VCP and remote control by pressing a button in the maintenance screen. It is programmed so that the aftertreatment regeneration can only be done in IDLE vehicle state, while the vehicle is guaranteed to be stationary. During the aftertreatment regeneration no other action can be performed on the machine, such as moving, tool operation, charging, etc. User should ensure that no persons are in the vicinity of the exhaust system since the exhaust temperatures can rise to even greater values than in normal engine operation. Start/stop button powers ON the base vehicle. After power-up the machine goes to IDLE state. In IDLE state the power system supplies auxiliary low-voltage to all of the systems and high-voltage to generator inverters and traction motor inverters. High-voltage is supplied to rest of the system when the machine is in OPERATIONAL state. The machine transitions into OPERATIONAL state when user presses the START button on either the base vehicle (VCP) or the remote controller and if no critical errors are pressed.

The base vehicle has following states that determine actions that it can perform:
OFFLINE state—This is state where vehicle is unpowered. Both the high and low voltage busses are not connected to vehicle electronics. The powering of the vehicle is started by pressing the START/STOP button. In order for vehicle to power up aux, battery voltage must be present.
IDLE state—This is the state that is entered when user presses the START/STOP button on unpowered vehicle. In this state the machine subsystems receive aux 24V power, but not high-voltage, with the exception of the battery charger and 700V/24V DC/DC converter. In this state the user can check machine state and read and set system parameters. Control of the machine movement, tools and mission module tools is not possible. In order to go to OPERATIONAL state user must issue a command via either VCP or OCU.
OPERATIONAL state—In this state the machine drivetrain and tools become operational. The machine can be thought of as being fully turned ON at this point. Every high-voltage DC bus becomes active after a few seconds of pre-charge procedure. In this mode user can fully control vehicle if the selected operating mode and hybrid mode allow it.
CHARGING state—In this state vehicle has 24V power and high voltage is provided only for battery charger and 700V/24V DC/DC converter. The vehicle is being charged and no movement of vehicle is possible. Only CRANE and STATIONARY operation modes are possible in this state.
ERROR state—In case any critical error occurs or user presses any of emergency stop buttons the vehicle transitions into this state. In this state every high voltage bus is disabled and parking brake is activated. Exiting this state is only possibly with powering OFF of the base vehicle.

Emergency stop button cuts power to all safety critical devices, i.e., devices whose continued operation might cause harm or damage to property.

These might include:
Traction motor inverters
Generator inverter
Battery pack contactors
Hydraulic pump inverter
Payload high voltage

In addition to cutting power to mentioned systems, emergency stop also does the following:
Activates parking brake by deactivating power supply of the brakes' hydraulic valve
Sends a signal to TRCU to activate mechanical brakes
Sends a signal to MBCU to go to EMERGENCY state

The base vehicle might comprise a warning system realised with a horn located at the back of the vehicle. The horn is activated manually by the user and automatically by the vehicle electronics in case faults are detected in machine's operation. These include:
Emergency stop switches activated
High-voltage isolation breach
Powertrain error
System overtemperature

The inlets for both diesel fuel and AdBlue are located on the left side of the machine behind a removable panel. The coolant oil inlets are located on the oil tanks in the Power Pack 400 in the rear compartment of the base vehicle. Coolant fluid tank is usually integrated into coolant system radiators in the back of the vehicle, on the Powerpack.

Base vehicle 100 has 4 auxiliary 12V batteries for supplying power during vehicle power-on, while the high voltage DCDC is not yet powered. Auxiliary batteries are also primary source of electrical power for diesel engine starter. Due to this, in case aux batteries are drained, power-up of the vehicle will not be possible without external power via NATO AUX socket. These auxiliary batteries are accessible within the panel on the right side of the vehicle.

In case the vehicle needs be towed the vehicle parking brake will need to be released. If electrical and subsequently the hydraulic power is not available, base vehicle features manual hydraulic pump for releasing the parking brake. Manual pump is located inside the panel on the left rear side of the vehicle.

In one embodiment the gripper 200 is with a blade and is attached to the front of the base vehicle. It uses rotating jaws and has lift capability of up to 2.000 kg. Gripper is equipped with lights and tool camera and is powered by a hydraulic pump within the base vehicle.

The manipulator arm 300 is positioned in the rear of the system, attached to the base vehicle. It comprises a swivel stand and three rotational arms. The swivel stand provides 360 deg non-continuous rotation. The manipulator arm is hydraulically operated. Energy for the manipulator arm is supplied from the base vehicle. The manipulator arm is detachable from the base vehicle. Hydraulic connection with the base vehicle and connection with the tool is equipped with hydraulic quick couplings. Hydraulic blocks are located on the manipulator arm. At the end of manipulator arm is attached special powered gripper tool comprising two additional rotating joints. Further on the manipulator arm are installed chemical warfare agents (CWA) and toxic industrial chemicals (TIC) detector, visual camera, shortwave and longwave thermal camera, temperature sensor and microphone. On the one rotational arm is integrated high pressure cleaning system and system for applying decontamination liquid. Water for high pressure cleaning and decontamination liquid is supplied from the mission module.

The manipulator arm 300 horizontal reach is 5 m, vertical reach for sampling is 7 m (height from ground level), also manipulator arm is able to decontaminate (from above) surfaces located up to 3.4 m above ground level, has a gripping tool with ability to grab objects with diameter up to 150 mm and lifting capacity up to 50 kg and manipulating capacity up to 25 kg.

Manipulator arm 300 is arranged so it collects samples of solid, liquid or gaseous matters what is performed by gripper tool. Sampling tubes are placed in the dedicated container located on the rear upper part of the mission module. Typically, container carries nine sampling tubes (three for each aggregate type of samples) and container is hermetically sealed and decontamination resistant.

Sampling procedure is automated in a way that a container lid opens and arm is positioned to take particular sampling tube. After sample is collected, contained in the tube, arm will return the tube into the bin and bin lid is closed.

The mission module is attachable/detachable to/from base vehicle via 4 connecting points, 2 at the front and 2 at the back of the module. After positioning, the mission module is secured with bolts on all 4 connecting points. Further the mission module is equipped with sprinklers mounted above the cooling air intake.

In one further embodiment user can activate prevention of entrance of contaminated atmosphere via operator control unit (OCU). When this mode is activated then the contaminated atmosphere cannot enter into the system, both base vehicle without engine compartment and the mission module. Air intake is filtered by CBRN filtration system that at the same time provides the necessary over-pressure. Before the vehicle enters the area with contaminated atmosphere, the user has to install CBRN filter in the filtration system. The lifetime of the CBRN filter is determined by the air pressure drop across it. When the pressure reaches a predetermined value, the filter is considered ready for replacement. CBRN filtration system measures this pressure drop and provides warning message on the OCU as it is approached.

In one embodiment the mission module might have electrical heaters built inside water tanks to prevent freezing of the water while the vehicle is exposed to water freezing temperatures. The power for the heaters is provided from the base vehicle. Further the mission module might be equipped with a firefighting system typically with following characteristics:
the firefighting system has a 1700 l water tank and 400 l foam tank positioned at the back of the mission module. In addition to water tank, the mission module is equipped with appropriate quick coupling interface for water intake from the outside source (hydrant, tank trailer, lake or other water sources)
water cannon with 2000 l/min flow rate, ±185° horizontal rotation, −45°/+90° vertical rotation and water range of 55 m (45 m with foam) is mounted on the stand positioned at the front end, outside of the mission module, this water cannon is equipped with an auto-regulated pressure nozzle with ability to adjust the shape of the water jet
the system is remotely controlled via OCU with an option of fire fighting with water or water/foam mixture with a foam dosage of 1%, 3% and 6%.
high pressure water pump is also a part of the firefighting system. The pump has a flow rate of 52 l/min and 150 bar working pressure. The mission module is equipped with appropriate interfaces (fast couplings) for transferring high pressure water to the manipulator arm and hand-held high-pressure water gun. The high-pressure water system for manipulator arm is remotely controlled via OCU, while the hand-held high-pressure water gun is enabled via OCU and then activated via VCP. Both systems can work simultaneously or separately.
as a part of the firefighting system CBRN mission module will have an option of self-protection against the high ambient temperature with water sprinkler system. Self-protection will remain functional even in case of individual nozzle malfunction.

Water for self-protection is supplied from the main water tank and is available as long as it is not turned off, or all the water has been used. OCU alarms the operator if any of the external ambient temperature sensors reads 65° C. Alarm might be in the form of warning message and warning sound. IRI_HMI software (part of Command-and-control Centre solution) has functionality to warn the operator by sound, show the measured value and position of the sensor on the graphical interface and log the data. Self-protection system includes an option of injecting water spray in the air intake of the base vehicle cooling system for additional heat dissipation. This functionality can be (de)activated simultaneously with self-protection from high temperature or (de)activated separately from it.

Further mission module is equipped with a decontamination system typically with following characteristics:
decontamination system has a 600 l tank positioned at the back of the mission module, with remotely controlled decontamination emulsion mixer. Besides that, 2 additional doses of decontamination agent are mounted on the mission module with a remotely controlled system for adding it to the decontamination tank
decontamination system involves multiple separate systems that can work independently. Those systems include: partial self-decontamination, decontamination with manipulator arm at the back of the vehicle, decontamination of the horizontal surfaces at the front of the vehicle, decontamination with shower at the back of the vehicle, decontamination with portable shower and hand-held decontamination gun. The showers and the hand-held decontamination gun are enabled via OCU and then can be activated via VCP, while all the rest of the system is remotely controlled via OCU only.
the decontamination system has a built-in heater that can heat up the decontamination emulsion up to 35° C. The fuel level of the heater tank is remotely monitored and the heater itself remotely controlled via OCU. The heater is remotely controlled and can be activated during vehicle operation.
partial self-decontamination system implies multiple decontamination nozzle network distributed on the mission module that can decontaminate the surface of the vehicle that is inaccessible for partial self-decontamination with manipulator arm. In combination with manipulator arm, decontamination nozzle network can decontaminate most of the vehicle's surface except the water cannon stand and the front tool which is decontaminated with hand held decontamination gun plugged to the mission module interface. The mission module is equipped with appropriate interfaces (fast couplings) for transferring decontamination emulsion to the manipulator arm and hand-held decontamination gun. Partial self-decontamination with manipulator arm is an automated process that only requires remotely controlled activation/deactivation from the user. Two automatic modes are present: partial self-decontamination of the vehicle tracks, partial self-decontamination of the vehicle outer shell.
the partial self-decontamination process implies washing with water from 20-130 bar of water pressure (necessary only if the surface is dirty or greasy) and dispersing the surface with the decontamination emulsion from 300-780 l/h.
Decontamination of the horizontal surfaces is a part of the decontamination system that is mounted on the front tool of the vehicle. The mission module is equipped with appropriate interfaces (fast couplings) for transferring decontamination emulsion to the front tool. Technical characteristics of decontamination of the horizontal surfaces include washing with water from 20-130 bar of water pressure (necessary only if the surface is dirty or greasy) and dispersing the surface with the decontamination emulsion from 300-780 l/h. Max vehicle speed, while decontaminating the horizontal surfaces, is 2-3 km/h and the minimum width of the decontamination trail is 2.3 m.
beside the partial self-decontamination, the manipulator arm is able to decontaminate buildings, fortifications and military/civilian machinery. The process includes washing with water from 20-130 bar of water pressure (necessary only if the surface is dirty or greasy) and dispersing the surface with the decontamination emulsion from 300-780 l/h.
decontamination system includes decontamination of the gear and clothing of the first response team with mounted shower at the back of the vehicle. Also, portable shower can be mounted at the safe distance from the vehicle and plugged to the appropriate interface (same coupling as the hand-held decontamination gun) for decontamination of the personnel. Both systems can be activated individually or at the same time.
powertrain space has an air filtration system located on the upper surface as a protection against contamination
after return from work in contaminated area the powertrain space will have to be decontaminated

Control of this vehicle and all of the subsystems is with hand held controller without integrated video. Hand held controller will display all feedback data from the vehicle needed for the operational use of it.

Control of this vehicle is implemented with the use of the command-and-control centre (CCC).

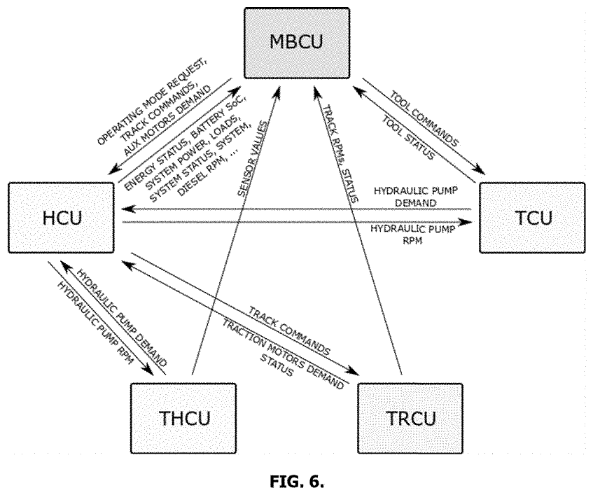

For the explaining for the functionality of the Remote Control this terminology is used:
Fast selection buttons—buttons that can be selected without interrupt on joystick input—as shown at FIG. 6.2
First level command—commands that can be selected in the one action, but can't be selected without interrupt on at least one joystick input (without releasing of the joystick)—FIG. 6.1 Keypad
Second level command—command that need to be combined with other command accessible on the same screen or keyboard
Third level command—command is accessible with changing of the screens (main menu needs to be invoked and different screen selected)

Remote Control system provides possibility to control the base vehicle, gripper, movement of the firefighting monitor, manipulator arm, firefighting system functions, decontamination system functions, camera system functions, mission module, power pack, autonomy and detection.

In one typical embodiment each of this controls is achieved as follows:
a) Base vehicle
   Movement of the driving joystick (left) provide input for the tracks of the machine
   Selecting of the driving gear scale position of the joystick to the demanded speed range (from 0 to the “selected gear*joystick position)
   Movement of the machine is dependent by the ‘System safety’ and ‘Deadman switch’ condition
   Selection of the “DeadMan” switch is by Fast selection button
   Selection of the “SystemSafety” switch is by First Level Command
   Activate Cruise control
   Function above is implemented as on MVF-5 Macro OCU
   Function below is implemented as Third level command
   Parking brake activation
   Silent mode (diesel engine activation disable)
b) Gripper (lift, tilt, grip, gripper pressure)
   Movement of the multifunctional (right) joystick provides input for the gripper movement
   Selecting of the gripper control is possible by selecting ‘gripper mode’ in the first level command
   Movement of the is dependent by the ‘System safety’ condition
   Function above is implemented as on MVF-5 Macro OCU
   Gripper Light is implemented by Second level command
c) Movement of the firefighting monitor (roll, pitch, height, focus)
   Movement of the multifunctional (right) joystick provides input for (roll, pitch, height) of the firefighting monitor movement
   Movement of the Z axis of the left joystick provides input for the focus of the firefighting monitor
   Selecting of the firefighting monitor control is possible by selecting ‘Monitor Mode’ in the first level command
   Movement of the firefighting monitor is dependent by the ‘System safety’ condition
d) Manipulator arm
   Controlling top of the arm in the normal operating mode (height of the tool selected calculated)
   Movement of the right joystick provides input for manipulator arm movement
   Selecting of the firefighting monitor control is possible by selecting ‘Manipulation Arm’ in the first level command
   Command for Manipulation arm decontamination is implemented as Second level command
   Command for Manipulation arm high pressure washing is implemented as Second level command
   Command for taking gripper tool is implemented as Second level command
   Command for taking water sampling tool is implemented as Third level command
   Command for taking soil sampling tool is implemented as Third level command
   Command for taking air sampling tool is implemented as Third level command
   Command for putting the tool in to the container is implemented as Third level command
   Command for arm parking is implemented as Second level command
For the controlling the arm in service mode this Third level function is implemented:
   Select arm first level control (right joystick controls base, first and second segment)
   Select arm second level control (right joystick controls arm third segment, top rotation and top tilt)
e) Firefighting system functions
   Water pump On/Off
   Pump regulation pressure/rpm
   Regulation of the firefighting pump pressure setting (active if pressure regulation is selected)
   Regulation of the firefighting pump rpm setting (active if rpm regulation is selected)
   Filling of the decontamination tank through water pump
   Automatic regulation of water level
   Pump intake from the water tank
   Pump intake from the decontamination tank
   Directing of the pressured water to cannon
   Mix foam
   Adjusting foam generation percentage (3-6%, regulate by 1%)
   Machine thermal self-protecting system (sprinkling of the water on the body of the machine
   Parking of the firefighting monitor
   Cleaning of the pipe system (after use of the firefighting foam or decontamination) Draining of the firefighting pump
   Draining of the water tank
   Use spare water level
   Selecting of the firefighting system tools is possible by the Second level command
f) decontamination system functions
   Selecting of functions below is possible by the Third level command
      Open decontamination powder container A
      Open decontamination powder container B
      Decontamination module on/off
      Mix decontamination liquid
      Partial machine self-decontamination
      Decontaminant liquid to firefighting pump
      Fill liquid from water tank to decontamination tank
   Selecting of the function below is possible by the Second level command
      decontamination of horizontal surfaces
      decontamination of the personnel (by the use of the shower)
g) Camera system functions
   Camera system provides selection of any camera attached on the machine by the Second level command and First level command
   First level commands are implemented by the use of right joystick and right First level buttons
   Access to camera menu is implemented by the Third level buttons
   Selecting of camera Iris/Focus is implemented by the Third level buttons
   Activating of camera wiper is implemented by Second Level command
   Function above is implemented as on MVF-5 Macro OCU
h) Mission module
   Function bellow is implemented as Third level command
      Activate Overpressure
      Activate Water installation heating
      Activate Water high pressure module
   Function bellow is implemented as First level command
      Front light
      Rear light
      Cannon light
i) Power pack
   Function bellow is implemented as Third level command
      Engine On
      Aftertreatment regeneration
j) Autonomy
   Function bellow is implemented as Third level command
      Allow ‘Return home”
      Allow ‘Patrolling’
k) Detection
   Function bellow is implemented as Third level command
      Activate LCD 3.3

For each element or subsystem Remote Control system has an ability to display following data:
b) Base vehicle
   Longitudinal inclination (pitch)
   Transversal inclination (roll)
   Autonomy actions active
   Gear (Mechanical)
   Parking brake active
   Hydraulic oil Level
   Speed
   Machine status
   Transmission system warnings
c) Gripper
   Lift position
   Tilt position
   Gripper rotate position
   Gripper grip position
d) Manipulator arm
   Arm parked
e) Firefighting system functions
   Water pump temp
   Water pump pressure
   Water pump RPM
   Foam pump RPM
   Water level
   Foam level
   Water intake pressure
   Water flow
   Foam flow
f) decontamination system functions
   Decontamination liquid Temp
   Decontamination liquid level
g) Camera system functions
   non
h) Mission Module
   Over pressure, pressure level
   Temp front left
   Temp front Right
   Temp Rear Left
   Temp Rear right
   Temp mast
i) Power pack
   Engine RPM
   Coolant temperature (×5)
   traction motors coolant tank temperature
   gearboxes coolant tank temperature
   left and right gearbox temperature sensors
   24V system voltage
   Engine Intake Air temp
   Engine Torque
   Engine status
   DC Bus High Voltage
   Battery status percent
   BMS Alarms
   Max Battery Temp
   Diesel fuel Level
   Aftertreatment Urea quality
   Aftertreatment temperature
   Aftertreatment Status
   Aftertreatment AdBlue level
   Battery current
   Generator torque
   Generator inverter faults
   Coolant system warnings
j) Autonomy
   Autonomy System ready
k) Detection
   LCD 3.3_A Agent1 identity
   LCD 3.3_A Agent2 identity
   LCD 3.3_A Agent3 identity
   LCD 3.3_A Agent4 identity
   LCD 3.3_A Agent1 Concentration
   LCD 3.3_A Agent2 Concentration
   LCD 3.3_A Agent3 Concentration
   LCD 3.3_A Agent4 Concentration
   LCD 3.3_A Agent1 accumulate dose
   LCD 3.3_A Agent2 accumulate dose
   LCD 3.3_A Agent3 accumulate dose
   LCD 3.3_A Agent4 accumulate dose
   LCD 3.3_B Agent1 identity
   LCD 3.3_B Agent2 identity
   LCD 3.3_B Agent3 identity
   LCD 3.3_B Agent4 identity
   LCD 3.3_B Agent1 Concentration
   LCD 3.3_B Agent2 Concentration
   LCD 3.3_B Agent3 Concentration
   LCD 3.3_B Agent4 Concentration
   LCD 3.3_B Agent1 accumulate dose
   LCD 3.3_B Agent2 accumulate dose
   LCD 3.3_B Agent3 accumulate dose
   LCD 3.3_B Agent4 accumulate dose
   RGR100 un filtrate dose
   RGR100 filtrate dose
   RGR100 accumulated dose
   Oxygen level
   Exp. Gasses level

Environmental sensorics and detection is divided in to point and ambient detection.

Point detection is done by:
Optical inspection by camera
Optical inspection by Longwave Thermal Imager
Optical inspection by Shortwave Thermal Imager
LCD 3.3 CWA & TIC detector
Temperature sensor
Sampling system that is realised by set of interchangeable tools that is attached and manipulated by the manipulation arm
Microphone that is installed on the top of the manipulation arm

Ambient detection is done by:
Polytron 5100 with Oxygen sensor
Polytron 5310Exp. Gasses detector
LCD 3.3 CWA & TIC detector
H3D radiological detector
GRAS 146AE Microphone
Set of temperature sensors installed on each corner of the mission module

The gas, aerosol and dust sampling tube comprises a housing and an electric motor with a blower, electronic tube operation control, sets of sample adsorption filters (cellulose filter, silicate filter and activated carbon filter) and outlet valves on the side of the housing and the bottom of the tube with the inlet valve. The lower part is detachable from the upper case. The tube is programmed and the filters are capacitated to filter from 0.5 m³ to 1 m³ atmosphere.

The sampling tube for liquid samples comprises a housing and an electric motor with electronic tube operation control, an electric motor gear, injector for the injection of fluid and the lower part of the tube with the inlet valve which is the sample carrier. The lower part is detachable from the upper case. Possible amount of liquid sample is up to 50 ml.

Solid sample sampling tube comprises a housing and an electric motor with electronic tube operation control, an electric motor gear, roller shafts and drill bits with pockets inside the roller to hold and hold the bulk and solid specimen, the lower part of the tube with the lid at the end for sealing the sample tube. The lower part is detachable from the upper case.

The container for housing the sampling tubes is located on the upper part of the superstructure, fastened to the superstructure, and the tubes in the crate are secured with supports. Usual capacity of the crate is 3×3 tubes, of which three tubes can be used to take a reference sample outside the contaminated area.

The whole system is arranged so that upon sampling task, the operator puts the robotic arm into mode for autonomously taking the tube from the crate after selecting the type of sample. After the tube is taken autonomously, the operator manipulates the robotic arm and brings the tube to the sampling point, and upon sampling, returns the robot arm to the mode for autonomously storing the tube in the crate.

In one embodiment, the control centre is implemented as set graphical user interfaces, video displays, machine operator work place and commander work place. Hardware is used for the communication and recording function and for the meteorological station that might be the part of control centre.

Typically, the following are the monitors and function that control centre consist of:
1) Display with telemetry from the machine
Function of this display is to provide GUI for the data that machine sends back to the CCS. Those data consist of telemetry from the machine with attached tools and data from the environmental sensors and detectors on the machine. To assist operator and commander with processing data that comes with the machine, dedicated software is developed (IRI_HMI).
IRI_HMI GUI will consist of 3D model of the machine with attached manipulator arm and readings from the environmental sensors and telemetry. To assist operator with processing of this data 3D model will present actual position of the machine (pitch and roll; data from the INS) and alarms if the feedback from the sensors exceeds alarm levels.
2) Display with video feedback from the machine
This display shows the feedback from the Dok-ing Rev.D Video system with PiP function.
3) Display with user interface for H3D Radiological Imager
Function of this display is to provide user interface and image from the Radiological Imager. User interface is in a form of web page provided by the sensors itself.
4) Display for QGIS (Geographic information system)
QGIS is used to present position of the machine on the map, position of the detections and as input interface for the autonomous driving
5) Display from Velodyne view (image from the LIDAR)
Display will show Velodyne view picture
6) Display for commander PC. This PC is for the purpose of the commander officer. PC has connection to the internet, SW for readings from the metrological station and prediction software.

In one further embodiment the vehicle provides trailer towing capability in a way that the vehicle is equipped with towing hook, max. mass of wheeled trailer is approx. 7 tons, max. vehicle speed would be approx. 10 km/h, vehicle is not allowed to move backwards and to turn on spot and there is no interface (electrical, pneumatic, . . . ) toward trailer. The base vehicle is equipped with hydraulic power pack and with tracks, each road wheel is independently suspended. The vehicle is equipped with working and parking brakes, usually braking deceleration is up to 5 m/s², parking brakes are of negative type.

The vehicle is capable to traverse water obstacles and is able to wade through 0.6 m deep water. The CBR resistance of the vehicle is provided by an overpressure system with filtration. Due to the necessity of using the surrounding atmosphere, powertrain (with Diesel engine and cooling system) is excluded from CBR contamination resistance. Also, powertrain is excluded from partial self-decontamination but powertrain space has separate air filtration.

If the mission module is detached from the base vehicle, it will only be possible to drive the base vehicle over the wire connection, with no additional information from the base vehicle. The base vehicle has any wireless communication module, it only requires external module for control which can either be a part of mission module (MM) or in case of no MM, a standalone control kit attached at the top of the vehicle or over a wired connection.

The vehicle is capable to detect explosive atmosphere and is able to detect and work in a reduced oxygen and oxygen free atmospheres.

In one further embodiment the front tool is designed as blade with the gripper and is hydraulically operated. The gripper might have continuous 360 rotation and regulated gripper pressure, does have 2.000 kg lifting capacity and 15 kN of gripping force and is equipped with lights and video system.

The rear tool is designed as detachable manipulator arm wherein arm is designed to provide sampling, detection and decontamination. For detecting there are integrated CWA and TIC detector, visual camera, shortwave and longwave thermal camera, temperature sensor and microphone.

Decontamination capability is provided by integration of high-pressure cleaning and system for applying decontamination liquid. Gripping tool has ability to grab objects with diameter up to 150 mm with lifting capacity up to 50 kg and manipulating capacity up to 25 kg.

In one further embodiment it is possible to implement autonomous control subsystem for controlling the base vehicle autonomously. It is designed as a modular system. The autonomous control subsystem consists of a control unit and sensors. Implemented functions are:
Return home
Waypoint following
Patrolling
Spatial mapping using LIDAR
Cartography in GPS-denied areas
Obstacle avoidance

Sensors used are GPS, 3D LIDAR and Inertial Measurement Unit (IMU) User is able to select in QGIS software a list of waypoints that the vehicle is supposed to reach autonomously. Due to possible high noise and drift in the GPS data, the waypoints are selected in global (GPS) coordinates while the navigation control loop will use local odometry and IMU.

Prerequisites for this function are that GPS has localization accuracy is under 1 m, IMU is present on the vehicle and the user must secure that there are no obstacles within 10 m of every waypoint. Equipment necessary for GPS way pointing function is GPS, IMU, navigation computer running ROS, computer running QGIS software and IP link between computers

For GPS patrolling user is able to select in QGIS software a list of waypoints that the vehicle is supposed to patrol along autonomously. Additionally, QGIS will store a waypoint of path travelled by the control of the operator and provide ability to use it as patrolling route. Prerequisites for this function are GPS has localization accuracy under 1 m, IMU is present on the vehicle and the user must secure that there are no obstacles on straight line paths between two adjacent waypoints.

Equipment necessary for GPS way-pointing function is GPS, IMU, navigation computer running ROS, computer running QGIS software and IP link between computers

With LIDAR Spatial Cartography user is able to obtain 3D point-cloud from the Velodyne LIDAR sensor. The point cloud is displayed on VeloView software running on computer that is part of Level-3 hardware of remote-control system.

Prerequisites for this function is IP link is present between the vehicle and control centre that has high enough throughput for LIDAR data and there is a following equipment necessary for GPS way pointing function: Velodyne LIDAR, computer connected to LIDAR onboard vehicle, computer running Veloview software and IP link between computers.

In relation to the LIDAR Spatial Cartography in GPS-denied areas, the vehicle is able to obtain 2.5 D map using LIDAR that vehicle can use for obstacle avoidance. 2.5 D is actually “Occupancy Gridmap”, where area is split into small predefined grids with occupancy value that corresponds to the highest obstacle in that grid area. This function uses odometry and IMU for localization. Resulting map is displayed on custom software running on computer that is part of Level-3 hardware of remote-control system. Prerequisites for this function is that terrain on which the vehicle is moving is flat

Equipment used for LIDAR Spatial Cartography in GPS-denied areas is Velodyne LIDAR, IMU, computer connected to LIDAR onboard vehicle, navigation computer running ROS, computer running visualization software and IP link between computers.

Upon loss of communication and if enabled by user input, the vehicle will attempt to return to the last known point with established communication link. To perform this home return function, it is required that the terrain on which the vehicle is moving is flat, GPS has localization accuracy under 1 m, IMU is present on the vehicle and user must secure that there are no obstacles on straight line paths between two adjacent waypoints. The necessary equipment for return home function is GPS, IMU, navigation computer running ROS, computer running QGIS software and IP link between computers.

Control subsystem implemented to the base vehicle comprises following control units:
Mobile Base Control Unit (MBCU)
Hybrid system Control Unit (HCU)
Transmission Control Unit (TRCU)
Thermal Control Unit (THCU)
Tool Control Unit (TCU)

Mobile Base Control Unit (MBCU) receives commands from user and relays appropriate commands to rest of control units. In addition to that it controls the general states of machine (OFFLINE, SILENT, DIESEL-ONLY and HYBRID MODE) and gathers telemetry data to send to the operator via RF link.

Hybrid System Control Unit (HCU) is the most complex of all the units in base vehicle. It takes care of the hybrid subsystem (diesel engine, generator, RESS, high-voltage power distribution) and controls high-voltage loads. It takes electrical power requirements obtained from other subsystems and selects the appropriate strategy depending on the user selected operating mode. The strategy consists of combining power flow from diesel-generator and to/from battery to satisfy power demand of the rest of the system. This also includes stopping some systems from using power if not enough power can be provided in selected mode for all the systems, for example when power is limited in SILENT mode due to the fact only battery power can be used.

Thermal Control Unit (THCU) controls the cooling subsystem units. This includes:
High-voltage power pumps
Low voltage power pumps
Cooling fans via hydraulic motors

THCU also communicates with temperature sensors and maintains temperatures of all the coolant circuits within allowed ranges while trying to consume as little power as possible. It is also monitoring coolant fluid levels and reporting to MBCU. In order to activate the fans, it needs to send request to HCU to spin the hydraulic pump to provide hydraulic power.

Transmission Control Unit (TRCU) controls the traction motors and transmission gearboxes operation. It monitors the demanded torque and speed of each track and implements automatic gear power-shifting algorithm. It does this via transmission subsystem's hydraulics. It combines torque from motor and friction of the clutches to achieve minimum shifting time with no apparent change of output velocity. In addition, it also controls the mechanical brakes.

Tool Control Unit (TCU) controls the hydraulic valves for the tool attachments. It receives commands from user that have been relayed from MBCU and activates the appropriate hydraulic valves. In order to activate the actuators, it needs to send request to HCU to spin the hydraulic pump to provide hydraulic power.

A system comprises claimed remote-controlled vehicle 10 and the control centre wherein control centre comprises a set of graphical user interfaces (GUI) and video displays (VD). Two-way communication between the vehicle 10 and control centre is established and from GUI the user can control and operate with the vehicle.

1. A remote-controlled vehicle for operations in the extreme conditions (10) characterised in that it comprises a base vehicle (100), a gripper (200), a manipulator arm (300), a mission module wherein base vehicle comprises base vehicle front part (110) and base vehicle rear part (120), wherein on the front part (110) of the base vehicle gripper (200) is mounted and on the rear part (120) of the base vehicle the manipulator arm (300) is mounted and when in operation, the gripper (200) and the manipulator arm (300) are remotely operated.

2. The remote-controlled vehicle (10) as claimed in claim 1, characterised in that it further comprises a sampling subsystem, environmental sensors and computed radiography (CR) detectors wherein the mission module is attached on top of the base vehicle (100), the sampling subsystem is attached partially to the manipulator arm (300) and partially to the mission module, and environmental sensors and CR detectors are installed partially on mission module and partially on manipulator arm (300).

3. The remote-controlled vehicle (10) as claimed in claim 1, characterised in that the mission module comprises a CBRN filter, water tanks, sprinklers and cooling air intake, wherein sprinklers are mounted above the cooling air intake and wherein when the claimed vehicle in operation water from water tanks is sprinkled over intake air in cooling air intake.

4. The remote-controlled vehicle (10) as claimed in claim 1, characterised in that the mission module further comprises electrical heaters built inside water tanks wherein the power for the heaters is provided from the base vehicle (100).

5. The remote-controlled vehicle (10) as claimed in claim 1, characterised in that the mission module further comprises a firefighting system, the said fire-fighting system further comprises a high pressure water pump, a water cannon and a fire-fighting water tank and a foam tank positioned at the back of the mission module, wherein said fire-fighting water tank is equipped with quick coupling interface for water intake from the outside source, the water cannon being equipped with an auto-regulated pressure nozzle with ability to adjust the shape of the water jet.

6. The remote-controlled vehicle (10) as claimed in claim 1, characterised in that the mission module further comprises a decontamination system comprising a decontamination emulsion mixer, at least one decontamination agent container and a tank positioned at the back of the mission module wherein the decontamination emulsion mixer a decontamination agent from decontamination agent container is remotely controlled.

7. The remote-controlled vehicle (10) as claimed in claim 1, characterised in that the mission module further comprises multiple decontamination nozzle network distributed on said mission module wherein, when claimed vehicle in operation, decontamination emulsion might be sprayed over the vehicle's exterior surfaces.

8. The remote-controlled vehicle (10) as claimed in claim 1, characterised in that the base vehicle (100) further comprises a hydraulic pump and the gripper (200) comprises rotating jaws, lights and tool camera wherein the rotating jaws are powered by the hydraulic pump within the base vehicle (100).

9. The remote-controlled vehicle (10) as claimed in claim 1, characterised in that the gripper (200) further comprises a blade.

10. The remote-controlled vehicle (10) as claimed in claim 1, characterised in that the manipulator arm (300) comprises a swivel stand and three rotational arms wherein swivel stand provides 360 deg non-continuous rotation and wherein the manipulator arm (300) is hydraulically operated.

11. The remote-controlled vehicle (10) as claimed in claim 1, characterised in that the manipulator arm (300) further comprises a gripper (200) tool attached at the end of the manipulator arm (300), wherein the gripper (200) tool is powered and comprises two additional rotating joints.

12. The remote-controlled vehicle (10) as claimed in claim 1, characterised in that the manipulator arm (300) further comprises chemical warfare agent (CWA) detector, toxic industrial chemicals (TIC) detector, visual camera, shortwave and longwave thermal camera, temperature sensor and microphone.

13. The remote-controlled vehicle (10) as claimed in claim 10, characterised in that one rotational arm comprises high pressure cleaning system and system for applying decontamination liquid.

14. The remote-controlled vehicle (10) as claimed in claim 2, characterised in that the sampling subsystem comprises at least one gas, aerosol and dust sampling tube or/and at least one sampling tube for liquid sample or/and at least one solid sample sampling tube wherein the gas, aerosol and dust sampling tube comprises a housing and an electric motor with a blower to generate pressure, an electronic tube operation control, sets of sample adsorption filters (cellulose filter o/and silicate filter or/and activated carbon filter) and outlet valves on the side of the housing and the bottom of the tube with the inlet valve, wherein when in operation blower creates air flow through the inlet valve and the adsorption of the sample on the sample adsorption filters and the sampling tube for liquid samples comprises a housing and an electric motor with electronic tube operation control, an electric motor gear, an injector for the injection of fluid and the lower part of the tube with the inlet valve, the lower part being detachable from the upper case, wherein when in operation upon completion of sampling executed by pressure generated by the injector, the inlet valve immobilizes the sample between the top piston of the injector and the inlet and solid sample sampling tube comprises a housing and an electric motor with electronic tube operation control, an electric motor gear, roller shafts and drill bits with pockets inside the roller, wherein said pockets hold bulk and solid specimen, the lower part of the tube with the lid at the end for sealing the sample tube, the lower part being detachable from the upper case, when in operations rotary drill drills and takes a sample wherein the lower part of the tube moves upwards by pressing the sample and opens the drill, and after completion spring action returns the drill to the basic position and closes the tube.

15. The system comprising the remote-controlled vehicle for operations in the extreme conditions (10) as claimed in claim 1 and a control centre wherein control centre comprises a set of graphical user interfaces (GUI) and video displays (VD) and wherein two-way communication between the vehicle and control centre is established and wherein from GUI person can control and operate with the vehicle.
